(19) United States
(12) Patent Application Publication
(10) Pub. No.: US 2009/0324759 A1
(43) Pub. Date: Dec. 31, 2009

(54) IMMUNE ENHANCEMENT BY SEED OIL AND/OR SEED FLOUR

(75) Inventors: Daniel A. Saltzman, Mendota Heights, MN (US); Arnold S. Leonard, Minneapolis, MN (US); Mark J. Mueller, Spooner, WI (US)

Correspondence Address: BRIGGS AND MORGAN P.A., 2200 IDS CENTER, 80 SOUTH 8TH ST, MINNEAPOLIS, MN, 55402, US

(73) Assignee: Botanic Oil Innovations, Inc., Spooner, WI (US)

(21) Appl. No.: 12/489,123

(22) Filed: Jun. 22, 2009

Related U.S. Application Data
(62) Division of application No. 11/220,473, filed on Sep. 7, 2005, now abandoned.

(51) Int. Cl. A61K 36/185; C12N 5/08; C12N 5/06; A61P 37/04

(52) U.S. Cl. 424/776; 435/372; 435/325; 435/349

(57) ABSTRACT

A method of treating a subject requiring immune mediation includes administering to the subject an immunostimulatory composition which contains black raspberry seed oil. Administering this composition stimulates the production of certain immune cells, including NK cells and CD8(+) lymphocytes. Administering the composition by this method also provides a method of treating, preventing, or ameliorating a condition or disease in the subject requiring enhanced immune system support.

### CROSS-REFERENCE TO RELATED APPLICATION

The present application is a divisional of and claims priority of U.S. patent application Ser. No. 11/220,472, filed Sep. 7, 2005.

### BACKGROUND OF THE INVENTION

The present invention relates to a composition useful as an immunostimulant and as an adjuvant to support various cancer treatment modalities. Additionally, this invention encompasses the use of a composition comprising seed oils and/or flours derived from the seeds and/or mixtures thereof as an immunostimulant and as a daily supplement to enhance the immune system activity of subjects.

Free radicals are atoms or molecules that are highly reactive with other cellular structures in the human body due to an unpaired electron. Consequently, free radicals are capable of chemically altering nearly all major classes of biomolecules (e.g., lipids, nucleic acids, proteins). Free radicals can be produced in vivo by factors such as dietary imbalances, tobacco smoke, pollutants, or from sources such as inflammation and biochemical reactions. They are capable of damaging DNA, inhibiting its repair, and increasing the probability of contracting a viral infections or cancer.

Interestingly, free radicals are also generated by cells of the immune system and their purpose is to destroy invading organisms. However, the presence of these strong oxidants places additional stress on the immune system (called “oxidative stress”), which can result in a diminished response against invaders. Furthermore, the long-term presence of these oxidizing species will eventually be detrimental to the human body. Oxidative stress is thought to play an important role in the pathogenesis of numerous chronic diseases, such as coronary heart disease and cancer. Although there are many factors in the development of these diseases, considerable experimental evidence has linked the production of free radicals to biological damage that can provide a basis for the initiation and progression of certain diseases.

Humans and other aerobic organisms have evolved a variety of mechanisms to protect themselves from the deleterious effects of free radicals. The body's defense systems, using certain enzymes, protect against free radical damage either directly or indirectly. Water and lipid-soluble antioxidants (such as ascorbate (vitamin C), alpha tocopherol (vitamin E), and beta carotene) also act to eliminate free radicals. These antioxidants help shield DNA from the deleterious effects of oxidative damage by absorbing unstable oxygen molecules.

A number of sources indicate a relationship between diet and cancer incidence in humans. The geographic distribution of certain types of cancer, the changing cancer patterns, and data from experimental animal studies all indicate that diet and nutrition are important factors in the control and prevention of human cancers. The potential importance of diet in cancer prevention is also noted by the suspected causes of some cancers. Most cancers (an estimated 80% to 90%) have environmental causes and are therefore potentially preventable. Much controversy surrounds the actual percent of cancers associated with dietary factors, but it has been estimated that in men 30% to 40% of all cancers are in some way related to diet. In women, it is believed that 60% o of all cancers are related to diet. Another study estimates that 35% of cancer is diet related. Regardless of the exact numbers, these are impressive percentages.

### Antioxidants

Presumably, pathology due to oxidative stress results when the generation of free radicals exceeds the cell's capacity to protect or repair itself. Therefore, if oxidative damage is an important etiologic factor in the pathogenesis of diseases such as cancer, then it follows that antioxidants, which act to reduce oxidative stress, may play a role in the prevention or treatment of these diseases. The accumulation and growth of free radicals in tissues is often found in association with suppressed immune function, including infections such as HPV and HIV, cancer, and heart disease. In fact, damage to heart blood vessels and the incidence of coronary heart disease has been shown to be reduced with increased dietary antioxidant intake. The protective effects of topical antioxidants (vitamin A derivatives such as retinoic acids) against proliferative dermatological diseases as well as photo-aging have been well documented. M any studies continue to demonstrate below normal antioxidant tissue and blood plasma levels in women with HPV and other cervical neoplasms, while high levels provide protection against their initiation and progression.

Like the B vitamins, the beneficial effects of antioxidants are most notable when combined with one another. In fact, diets high in antioxidants (e.g., the traditional Greek Mediterranean diet) have been shown to be protective against cancer and various diseases. It is believed that antioxidants alter cancer incidence and growth by acting as anti-carcinogens. Nutritional anti-carcinogens function by (a) inhibition of tumor initiation via alteration of cellular metabolism, (b) picking up active forms of carcinogens and preventing them from reaching target sites, (c) alteration of the body's defense systems, (d) inhibition of cancer progression once it has been initiated by the alteration of cell differentiation, and (e) prevention of gene activation and cellular proliferation by tumor promoters.

### Plant Seed Oils

Plant seed oils that can be extracted from the fruit, leaves, or seeds of various plants have been found to be an excellent source of antioxidants. In addition to traditional antioxidants such as vitamins C and E, plant oils contain phenolic compounds which are excellent free radical scavengers due to their electron structure. Recent studies have shown that plant oils rich in antioxidants also increase cellular immune response. During the past three decades immunotherapy has become an important approach for treating human diseases and conditions through the use of regimens designed to modulate immune responses. This is particularly important in pathological conditions where the immune system becomes compromised. Studies conducted in disease models and clinical trials demonstrate that augmenting host defense mechanisms is useful in treatment and prophylaxis against microbial infections, immunodeficiencies, cancer, and autoimmune disorders. Immune enhancing protocols may also have utility for promoting wound healing. In the process of wound healing, macrophages exhibit a principal role by modulating cellular proliferation and new tissue formation/regeneration. They also function as phagocytes, debridement agents and produce growth factors that influence the angiogenesis stage of wound repair.

Although current immunostimulants show promise, there is still a need to develop more potent agents and increase the arsenal of available drugs for immunotherapy. One source of chemically diverse compounds that can be used for drug discovery of immunostimulants is natural products. For centuries natural products have been exploited as therapeutically useful agents, many of which are in clinical use today. Interest in natural products as a means to drug discovery is based on their unparalleled molecular diversity and rich spectrum of biological activities.

### BRIEF SUMMARY OF THE INVENTION

The present invention relates to a composition comprising an oil derived from black raspberry seeds processed utilizing technology to optimize the yield of nutrient-dense food grade oil each seed contains. Black raspberry and black cumin seeds are diverse sources of healthy nutrients including Omega 3 & 6 essential fatty acids, potent antioxidants (vitamin E tocopherols and tocotrienols among others) and other health promoting phytochemicals. The present composition provides nutritional support of the immune system. Hence, this invention contemplates the use of the seed oil composition as a daily supplement, a immunostimulant and as an adjuvant.

Black Cumin (Nigella sativa) has been consumed in the Middle East and Asia for more than a thousand years. The diverse health promoting properties of this special botanical have been the subject of numerous scientific studies and clinical trials including its beneficial impact on the immune system. Its antioxidant level is among the very highest of all plant oils, and therefore it has strong free radical scavenging activity (quenching undesirable substances that disrupt healthy cells). It is known that free radicals are generated during aging and some disease states.

Black and Red Raspberry Seed Oils have a diversity and ultra-rich content of antioxidants, including 4 different forms of Vitamin E (Alpha and Gamma Tocopherol, Beta and Gamma Tocotrienol). They also contains abundant Omega 3 and Omega 6 essential fatty acids. These fatty acids are essential to maintaining optimum health because they are used as building blocks for hormones that regulate many vital health functions (“essential” because the body cannot produce them on its own and they must be obtained from the daily diet).

The instant invention comprises oil derived from black raspberry seeds and can further comprise other oils and/or flours derived from red raspberry seed, black cumin seed, evening primrose seed, cranberry seed, mullein seed, parsley seed, carrot seed, blueberry seed, chardonnay grape seed, merlot grape seed oils. The blend provides an exceptionally nutritious health promoting food supplement created to provide positive nutritional support for a broad array of health situations and dietary requirements.

The oils for the composition are prepared from seeds which have been carefully dried and cleaned at temperatures below 120 degrees F. In a cold press process, the seeds are fed through the press and put under high pressure with no extra heat during the pressing process. Oil temperatures during extraction are typically 70 degrees to 90 degrees F. To insure minimal or no oxidation and the highest potential antioxidant level of the oils, the press head and oil extraction chamber can be enclosed within an inert atmosphere. Refining or removal of suspended solids and container filling can also be done in an inert atmosphere to preserve quality.

The inventors have found that the instant oil composition boosted the immune system by encouraging the population growth of CD 8 white lymphocytes and NK killers cells. The CD 8 white blood cells are known to identify foreign invaders in the body such as viruses, infections and tumors and the NK cells attack the foreign invaders. A significant increase in NK killer cells and CD 8 white lymphocytes was observed within seventy two hours.

The instant oil composition can also be used as an adjunct treatment for immunomodulation specific to cancer for example in the treatment of hepatic colorectal cancer and neurobalstomas.

According to another embodiment, a method of enhancing immune function in an individual in need of such treatment, comprises administering to said individual an effective amount of the present seed oil containing pharmaceutical composition or dietary supplement. According to another embodiment, the individual is suffering from a viral, bacterial or fungal infection. According to another embodiment, the individual is suffering from cancer. According to another embodiment, the individual is suffering from an immune deficiency. According to another embodiment, the individual is a human being. According to another embodiment, the individual is an animal.

According to another embodiment, a method of treating an individual with an immunostimulatory seed oil preparation in order to provide to the individual a stimulation of monocyte/macrophage activity comprises administering to the individual an effective amount of the present seed oil preparation in combination with an acceptable carrier. According to another embodiment, the immunostimulatory seed oil preparation is administered to enhance wound healing via topically applied preparations containing the oils. According to another embodiment, the immunostimulatory seed oil preparation is administered as an adjunct to treat cancer. According to another embodiment, the immunostimulatory seed oil preparation is administered to treat immunodeficiency.

According to another embodiment, the immunostimulatory seed oil preparation is administered to treat a viral, bacterial or fungal infections. According to another embodiment, the individual is a human being. According to another embodiment, the individual is an animal. According to another embodiment, a method of treating an individual with an immunostimulatory seed oil preparation in order to provide to the individual a stimulation of monocyte/macrophage activity comprises administering to the individual an effective amount of a seed oil preparation in combination with an acceptable carrier.

### DETAILED DESCRIPTION OF THE INVENTION

For over a decade, we have been investigating methods to treat liver cancer (adenocarcinoma that metastasized from the colon) in mice in our laboratory. We postulated that enhancing the mouse's immune system in part with antioxidants may improve clinical success of the cancer treatment methodology we were researching. Certain plant seed oils are extraordinarily high in antioxidants and, for the purpose of our study, black raspberry seed oil was used as a dietary supplement to provide the mice with a source of a variety of antioxidants because it has one of the highest levels of antioxidants. Moreover, black raspberry seed oil has a higher oxygen radical-reducing capacity than palm, corn, sunflower or olive oil.

The oil was obtained from the black raspberry seeds via the cold press manufacturing method. The cold press extraction method yields excellent quality oil with minimal chemical decomposition compared to traditional high temperature extraction methods. When using cold-pressing, nothing is added to the seeds or to the oil to increase production during the manufacturing process. The oils are pure and unadulterated.

In the study, black raspberry seed oil was added daily to the food of the mice and the amount of oil administered was equal to 10% of the rodent's weight. After varying periods of time, the mice were euthanized and their body chemistry carefully examined.

In the course of this research, it was discovered that the highly potent antioxidant oil, black raspberry seed oil, appeared to augment the populations of both NK (natural killer cells) and CD8(+) T (cytotoxic T lymphocyte) cells. This research is key because an increase in the number of NK and CD8(+) cells enables to body to better fight the damage caused by free radicals and enhance the body's ability to fight disease and cancer.

### The Study

Evaluation of Splenic Lymphocyte Populations when Black Raspberry Seed Oil is Administered

### Experimental Design:

4 groups of mice were used at 6-8 weeks of age

### Day 0

Group 1—standard rodent meal
Group 2—standard rodent meal+10% black raspberry oil

### Day 3, 7, or 14

Mice were sacrificed for spleen harvest and to obtain cardiac blood samples. Spleens were processed for flowcore analysis and the data processed using StatView. Cardiac blood was centrifuged to collect serum for ELISA analysis of circulating cytokines at a later date.

### Results/Summary:

Recent studies have shown that plant oils rich in antioxidants also increase cellular immune response. We postulated that the addition of antioxidant oil to a murine diet hypothesis, animals were randomly placed into two groups: diet with and without oil. For the duration of each study, the animals were fed standard rodent chow or standard rodent chow supplemented with 10% (by weight) black raspberry seed oil, a potent antioxidant plant oil. Splenic lymphocyte populations were analyzed 3, 7, and 14 days. We found significantly elevated CD8(+) lymphocyte populations in the oil group at all time points when compared to the other group. Furthermore, we observed that the NK population peaked on day 7. These results suggest that antioxidant oil in conjunction with the normal diet augments cytotoxic immune response.

EXPERIMENTAL DESIGN

| | Without Oil | With Oil |
|---|---|---|
| Diet | Standard rodent diet | Standard rodent diet + 10% |
| | | black raspberry seed oil by |
| | | weight |

FIG. 1 shows the effect of black raspberry seed oil diet on natural killer (NK) cell populations. After splenocytes were isolated, flow cytometry was utilized to determine the percentage of NK cells present. Each value represents the mean+SD of at least 5 mice. Error bars are not apparent if less than 0.5.

FIG. 2 shows the effect of black raspberry seed oil diet on cytotoxic T lymphocyte (CD8+) populations. After splenocytes were isolated, flow cytometry was utilized to determine the percentage of CD8(+) lymphocytes present. Each value represents the mean±SD of at least 5 mice. Error bars are not apparent if less than 0.5.

In experiments performed at the University of Minnesota Medical School, Black Raspberry Seed Oil was used in conjunction with a form of Salmonella that does not cause disease and which has been genetically altered to carry a gene for a powerful immune stimulant. It is known that Salmonella readily invades tumor tissue and we have successfully used the organism to deliver the immune stimulant. Combining the Salmonella with Black Raspberry Oil further enhances our immune stimulation and tumor destruction in mice with liver cancers.

FIG. 3 is an example of an experiment showing tumor reduction in a two week period using the potent antioxidant oil alone and in combination with the Salmonella carry a gene for immune stimulant.

### Pharmaceutical Formulations

Since the present seed oil preparations maybe useful as agents for immunotherapy in the treatment of immunodeficiency disorders, cancer, wound healing and infectious diseases, the present invention includes pharmaceutical compositions containing the instant seed oil preparations optionally in combination with acceptable pharmaceutical carriers or excipients.

Pharmaceutical compositions suitable for use in the present invention include compositions wherein the active ingredients are contained in an effective amount to achieve its intended purpose. More specifically, a therapeutically effective amount means an amount effective to prevent development of or to alleviate the existing symptoms of the subject being treated. Determination of the effective amounts is well within the capability of those skilled in the art, especially in light of the detailed disclosure provided herein.

The amount of composition administered will be dependent upon the condition being treated, the subject being treated, on the subject's weight, the severity of the affliction, the manner of administration and the judgment of the personalizing physician.

The pharmaceutical compositions of the present invention may be manufactured in a manner that is itself known, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries which facilitate processing of the compositions compounds into preparation which can be used pharmaceutically. Proper formulation is dependent upon the route of administration chosen.

For injection, the agents of the invention may be formulated in aqueous solutions, preferably in physiologically compatible buffers such as Hanks solution, Ringer's solution, or physiological saline buffer. For transmucosal administration, penetrants appropriate to the barrier to be permeated are used in the formulation. Such penetrants are generally known in the art.

For oral administration, the compositions can be formulated readily by combining the active compositions with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained as a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethyl cellulose, and/or polyvinylpyrrolidone (PVP).

If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as fit, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compositions for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a power mix of the compound and a suitable powder base such as lactose or starch.

The compositions may be formulated for parenteral administration by injection, e.g., by bolus injection or continuous infusion. Formulations for injection may be presented in unit dosage form, e.g., in ampoules or in multidose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active composition may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compositions may also be formulated in rectal compositions such as suppositories or retention enemas, e.g., containing conventional suppository bases such as cocoa butter or other glycerides.

In addition to the formulations described previously, the compositions may also be formulated as a depot preparation. Such long acting formulations may be administered by implantation (for example subcutaneously or intramuscularly) or by intramuscular injection. Thus, for example, the compositions may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable routes of administration may, for example, include oral, rectal, transmucosal, transdermal, or intestinal administration, parenteral delivery, including intramuscular, subcutaneous, intramedullary injections, as well as intrathecal, direct intraventricular, intravenous, intraperitoneal, intranasal, or intraocular injections.

Alternatively, one may administer the composition in a local rather than systemic manner, for example, via injection of the compound directly into an affected area, often in a depot or sustained release formulation.

Furthermore, one may administer the drug in a targeted drug delivery system, for example, in a liposome coated with an antibody specific for affected cells. The liposomes will be targeted to and taken up selectively by the cells.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. Compositions comprising a composition of the invention formulated in a compatible pharmaceutical carrier may also be prepared, placed in an appropriate container, and labeled for treatment of an indicated condition. Suitable conditions indicated on the label may include treatment of a disease.

### Dietary Supplements

Dietary supplements suitable for use in the present invention include compositions wherein the active ingredients are contained in an effective amount to achieve its intended purpose. More specifically, an effective amount means an amount effective to prevent development of or to alleviate the existing symptoms of the subject being treated. Determination of the effective amounts is well within the capability of those skilled in the art, especially in light of the detailed disclosure provided herein. The amount of composition administered will be dependent upon the condition being treated, the subject being treated, on the subjects weight, the severity of the affliction, the manner of administration and the judgment of the personalizing physician.

The ingredients of the dietary supplement of this invention are contained in acceptable excipients and/or carriers for oral consumption. The actual form of the carrier, and thus, the dietary supplement itself, may not be critical. The carrier may be a liquid, gel, gelcap, capsule, powder, solid tablet (coated or non-coated), tea or the like. Suitable excipient and/or carriers include maltodextrin, calcium carbonate, dicalcium phosphate, tricalcium phosphate, microcrystalline cellulose, dextrose, rice flour, magnesium stearate, stearic acid, croscarmellose sodium, sodium starch glycolate, crospovidone, sucrose, vegetable gums, agar, stevia, lactose, methylcellulose, povidone, carboxymethylcellulose, corn starch, and the like (including mixtures thereof). The various ingredients and the excipient and/or carrier are mixed and formed into the desired form using conventional techniques. Dose levels/unit can be adjusted to provide the recommended levels of ingredients per day in a reasonable number of units.

The dietary supplement may also contain optional ingredients including, for example, herbs, vitamins, minerals, enhancers, colorants, sweeteners, flavorants, inert ingredients, and the like. Such optional ingredients may be either naturally occurring or concentrated forms.

Selection of one or several of these ingredients is a matter of formulation, design, consumer preference and end-user. The amounts of these ingredients added to the dietary supplements of this invention are readily known to the skilled artisan. Guidance to such amounts can be provided by the U.S. RDA doses for children and adults.

### LITERATURE

Di Mascio P D, Murphy M E, Sies H. Antioxidant defense systems: the role of carotenoids, tocopherols and thiols. Am J Clin Nutr 53:1945, 1991.
Freeman B A, Crapo J D. Biology of disease: free radicals and tissue injury. Lab Investig 47:412, 1982.
Slater T F, Cheeseman K H, Davies M J, Proudfoot K, Xin W. Free radical mechanisms in relation to tissue injury. Proc Nutr Soc 46:1, 1987.
Chan A C, Chow C K, Chiu D. Interaction of antioxidants and their implication in genetic anemia. Proc Soc Exp Biol Med 222:274, 1999.
Kwasniewska A, Tukendorf A, Semczuk M. Content of alpha-tocopherol in blood serum of human papillomavirus-infected women with cervical dysplasias. Nutr Cancer 28:248, 1997.
Yu B P. Cellular defenses against damage from reactive oxidative species. Physiol Rev 74:139, 1994.
Halliwell B, Chirico S. Lipid peroxidation: its mechanism, measurement, and significance. Am J Clin Nutr 57:7155, 1993.
Loft S, Poulsen H E. Cancer risk and oxidative DNA damage in man. J Mol Med 74:297, 1996.
Stahl W, Sies H. Antioxidant defense: vitamins E and C and cartenoids. Diabetes 46: S 14, 1997.
Keaney J F Jr., Frei B. Antioxidant protection of low-density lipoprotein and its role in the prevention of atherosclerotic vascular disease. In: Frei B, ed. Natural antioxidants in human health and disease. New York: Academic Press, 1994: 303.
Romney S L, Palan P R, Basu J, Mikhail M. Nutrient antioxidants in the pathogenesis and prevention of cervical dysplasias and cancer. J Cell Biochem Suppl 23:96, 1995.
Lyon J L, Klauber M R, Gardner J W, Smart C R. Cancer incidence in Mormons and non-Mormons in Utah, 1966-1970. New Engl J Med 194:129, 1976.
Newberne P M, Suphakarn V. Nutrition and Cancer: A review with emphasis on the role of vitamins C and E and selenium. Nutr Cancer 5:107, 1983.
Diet and cancer: What do we know, so far? Your Patient and Cancer 2:49, 1982.
White K. Diet and cancer. Med World News 23:52, 1982.
Newell, G. R. The provocative tale of diet in carcinogenesis. Consultant 24:116, 1984.
Weisburger J H. Antimutagenesis and anticarcinogenesis, from the past to the future. Mutat Res 480:23, 2001.
Ahmed M I, Fayed S T, Hossein H, Tash F M. Lipid peroxidation and antioxidant status in human cervical carcinoma. Dis Markers 15:283, 1999.
Feher J, Blazovics A, Somogyi A, Lengyel G. The role of oxidative stress and the preventive effect of free radical scavengers in arteriosclerosis. Orv Hetil 138:2283, 1997.
Favier A, Sappey C, Leclerc P, Faure P, Micoud M. Antioxidant status and lipid peroxidation in patients infected with HIV. Chem Biol Interact 91:165, 1994.
Maxwell S R. Coronary artery disease—free radical damage, antioxidant protection and the role of homocysteine. Basic Res Cardiol 95:165, 2000.
Dreher F, Maibach H. Protective effects of topical antioxidants in humans. Curr Probl Dermatol 29:157, 2001.
Henry F, Claessens N, Martalo O, Fraiture A L, Pierard-Franchimont C, Pierard G E. Towards obsolete senescence. Everything wanes . . . . Old age no longer exists! Rev Med Liege 55:110, 2000.
Nagpal 5, Chandraratna R A. Vitamin A and regulation of gene expression. Curr Opin Clin Nutr Metab Care 1:341, 1998.
Goodman M T, Kiviat N, McDuffie K, Hankin J H, Hernandez B, Wilkens L R, Franke A, Kuypers J, Kolonel L N, Nakamura J, Ing G, Branch B, Bertram C C, Kamemoto L, Sharma S, Killeen J. The association of plasma micronutrients with the risk of cervical dysplasia in Hawaii. Cancer Epidemiol Biomarkers Prey 7:537, 1998.
Liu T, Soong S J, Wilson N P, Craig C B, Cole P, Macaluso M, Butterworth C E Jr. A case control study of nutritional factors and cervical dysplasia. Cancer Epidemiol Biomarkers Prey 2:525, 1993.
Trichopoulou A, Vasilopoulou E. Mediterranean diet and longevity. Br J Nutr 84:S205, 2000.
Hosack-Fowler K, Chapkin R S, McMurray D N. Effects of purified dietary n−3 ethyl esters on murine T lymphocyte function. J Immunol 151:5186, 1993.
Calder P C. Dietary fatty acids and the immune system. Nutr Rev 56: S70, 1998.
DeWille J W, Fraker P J, Romsos D R. Effects of essential fatty acid deficiency and various levels of dietary polyunsaturated fatty acids on humoral immunity in mice. J Nutr 109:1018, 1979.
Fritsche K L, Johnston P V. Effect of dietary omega-3 fatty acids on cell-mediated cytotoxic activity in BALB/C mice. Nutrition Research 10:577, 1990.
Sionek B. Cold pressed oils. Roczniki Panstwowego Zakladu Higieny 43:283, 1997.
Hillyer L M, Woodward B. A comparison of the capacity of six cold-pressed plant oils to support development of acquired immune competence in the weanling mouse: superiority of low-linoleic-acid oils. Br J Nutr 88:171, 2002.
Blumgart L H, Fong Y. Surgical options in the treatment of hepatic metastasis from colorectal cancer. Curr Probl Surg 32:333, 1995.

We claim:

1. A method of treating a subject requiring immune mediation comprising administering to said subject an immunostimulatory composition that comprises a seed oil preparation obtained from black raspberry seed, Rubus occidentalis, the immunostimulatory composition increasing the population of splenic NK and CD8(+) lymphocytes.

2. A method of activating an immune cell having a receptor that recognizes a constituent of the immunostimulatory composition of claim 1 in a subject, comprising: providing an effective immune cell activating amount of the immunostimulatory composition of claim 1 and introducing said preparation to said subject.

3. The method of claim 2, wherein the immune cell is a monocyte.

4. A method of treating, preventing, or ameliorating a condition or disease in a subject requiring enhanced immune system support; comprising providing an effective immune cell activating amount of the composition of claim 1, and administering said composition to said subject.

5. The method of claim 4, wherein the condition or disease is an immune deficiency.

6. The method of claim 4, wherein the condition or disease is cancer.

7. The method of claim 4, wherein the condition or disease is a fungal, viral, or bacterial infection.

8. The method of claim 4, wherein the condition or disease is a wound.

9. The method of claim 4, wherein the subject is a human.

10. The method of claim 4, wherein the subject is a mammal, reptile, fish, pet, bird, domesticated animal, farm animal, animal or other living organism.

11. A method of activating an immune cell having a receptor that recognizes a constituent of an immunostimulatory composition comprising black raspberry seed Rubus occidentalis oil, comprising: providing an effective immune cell activating amount of the immunostimulatory composition and introducing said composition to a subject.

12. The method of claim 11, wherein the immune cell further comprises an NK cell.

13. The method of claim 12, wherein a population of NK cells in the subject as a percent of all splenocytes increased by about 1% over the population of NK cells as a percent of all splenocytes in a subject not receiving the immunostimulatory composition.

14. The method of claim 11, wherein the immune cell further comprises a CD8(+) cell.

15. The method of claim 14, wherein a population of CD8(+) cells in the subject as a percent of all splenocytes increased by about 1% to 2% over the population of CD8(+) cells as a percent of all splenocytes in a subject not receiving the immunostimulatory composition.

16. A method of treating, preventing, or ameliorating a condition or disease in a subject requiring enhanced immune system support, comprising providing an effective immune cell activating amount of a composition comprising black raspberry seed Rubus occidentalis oil, and administering said composition to said subject, wherein said composition increases the population of immune cells in the subject.

17. The method of claim 16, wherein the immune cells further comprise NK cells.

18. The method of claim 17, wherein a population of NK cells in the subject as a percent of all splenocytes increased by about 1% over the population of NK cells as a percent of all splenocytes in a subject not receiving the immunostimulatory composition.

19. The method of claim 16, wherein the immune cells further comprise CD8(+) cells.

20. The method of claim 19, wherein a population of CD8(+) cells in the subject as a percent of all splenocytes increased by about 1% to 2% over the population of CD8(+) cells as a percent of all splenocytes in a subject not receiving the immunostimulatory composition.
